(19) United States
(12) Patent Application Publication
(10) Pub. No.: US 2018/0325795 A1
(43) Pub. Date: Nov. 15, 2018

(54) MUCIN TOPICAL DEODORANTS AND ANTIPERSPIRANTS

(71) Applicant: NATURA COSMÉTICOS S.A., São Paulo (BR)

(72) Inventors: Renata HANNEL BUELONI, São Paulo (BR); Roberta ROESLER, São Paulo (BR); Joice PANZARIN SAVIETTO, São Paulo (BR)

(21) Appl. No.: 15/774,164

(22) PCT Filed: Nov. 7, 2016
(86) PCT No.: PCT/BR2016/050284

§ 371 Date: May 7, 2018

Related U.S. Application Data
(60) Provisional application No. 62/251,420, filed on Nov. 5, 2015.

(51) Int. Cl. A61K 8/64; A61K 8/02; A61K 8/04; A61K 8/06; A61Q 15/00

(52) U.S. Cl. CPC A61K 8/64; A61K 8/0229; A61K 8/042; A61K 8/046; A61K 2800/874; A61Q 15/00; A61K 2800/30; A61K 2800/524; A61K 8/06

(57) ABSTRACT

The present invention relates to aluminum-containing and aluminum-free mucin topical deodorants and antiperspirants. It is an objective of this invention to provide a topical deodorant and/or antiperspirant formulation for suppressing emergence of body odor formation, comprising a mucin and a physiologically acceptable carrier, which is particularly free of aluminum-containing substances. It is a further objective of this invention to provide the use of a mucin in the preparation of a topical deodorant and/or antiperspirant formulation for suppressing emergence of body odor formation, said formulation being particularly free of aluminum-containing substances.

### FIELD OF THE INVENTION

The present invention is comprised within the field of cosmetic and personal care industries, particularly deodorants and antiperspirants for the human body.

### BACKGROUND OF THE INVENTION

Deodorants:

A deodorant is a substance that is used to eliminate bad odor which is generally caused by sweating. Odor is generated from secretions of the apocrine sweat glands, which are primarily contaminated by Coryneform and Staphylococcus bacteria, as for example C. jeikeium, C. striatum, C. xerosis, S. epidermis and S. haemolyticus. C3-fatty acids, (iso)butyric acid, isovaleric acid, and androgen steroids have been exemplified as the odorous substances. Deodorants are mostly applied locally on surface of the body. Many chemical compounds are used to make a deodorant, which can also contain antiseptics or specific agents that destroy or prevent bacteria, which is responsible for metabolizing proteins and fatty acids.

One of the utmost advantages of deodorants is that it has the capability to manage the odor by neutralizing it. A deodorant has that ability to protect the body from excessive sweat, and eliminates bacteria that causes bad odor. Deodorants are preferred more than antiperspirants as it has proved a fact that deodorants do not prevent sweating unnaturally. There are different kinds of deodorants containing scents from floral, sporty and the breezy ones. Besides these choices, there are some deodorants which have no scents but works perfectly in neutralizing the awful odor. People can even try making their own home made deodorants by using natural ingredients that pamper the skin and give a feeling of freshness throughout the day.

Deodorants are used to control the body odor, but when you use deodorants, some of them have side-effects on clothes and skin. These side effects are for example decreases in perspiration: aluminum-containing deodorants block sweat ducts (sudoriparous ducts), and this can build up toxins in our armpits. Aluminum-containing substances also can buildup estrogen, which causes breast cancer.

Antiperspirants:

An antiperspirant is a substance which is used to prevent or reduce sweating through blocking the pores by using for example aluminum, effectively changing the function of the body. Without sweat, the bacteria cannot metabolize proteins and fatty acids that cause body odor. Antiperspirants have some side-effects like: rashes and skin irritations but they can be easily treated—the use of antiperspirants on broken skin is not advisable as they can cause blood poisoning.

Antiperspirants have a certain fragrance, which has a capability to prevent sweat and to neutralize the unpleasant odor. Antiperspirants block the pores with the help of chemical substances, thus controlling sweating. Antiperspirants can contain aluminum, which helps clogging the pores and it also prevents the sweat spots. Antiperspirants usually have their effects for 12 or 48 hours. However, the use of excessive antiperspirants can also cause complications and, therefore, it is advised that antiperspirants are used sparsely.

It is known in the art that antiperspirants are used for controlling sweat. However, there are some side effects that people must be aware of, as for example lumps: some antiperspirants block the pore of the skin and cause bacterial infections like armpits' lumps. Aluminum-containing antiperspirants can also cause breast cancer.

Currently, there are deodorants combined with antiperspirants, and vice-versa.

Aluminum:

Most aluminum compounds currently used in cosmetics are exemplified as follows: aluminium starch octenylsuccinate, aluminum chlorohydrate, aluminum hydroxide, aluminum chloride and aluminum-zirconium compounds.

Aluminum hydroxide is used widely in antiperspirants, and as a filler in cosmetics. Aluminum starch octenylsuccinate is used in cosmetic formulations as anti-caking and viscosity-increasing agents. Aluminum chloride, aluminum chlorohydrate and aluminum-zirconium compounds, most notably aluminum zirconium tetrachlorohydrex gly and aluminum zirconium trichlorohydrex gly, are the most widely used in antiperspirants.

Aluminum salts can account for 25% of the volume of some antiperspirants, and common sources of aluminum exposure for humans show that antiperspirant use can significantly increase the amount of aluminum absorbed by the human body. It is known that, after a single underarm application of antiperspirant, about 0.012% of aluminum is absorbed by the body.

At high doses, aluminum itself adversely affects the blood-brain barrier, is capable of causing DNA damage, and has adverse epigenetic effects. The absorption of aluminum through the skin can cause a greater burden on the body than oral ingestion. After using aluminum-containing antiperspirants, in case of aluminum absorption by the body, it is known that aluminum can still be present in the blood 15 days after one application of said aluminum-containing antiperspirants to the armpit. Consequently, applying aluminum to the skin is a very effective way to get aluminum in the human blood system, and the brain. Aluminum species used in cosmetics can cause a series of diseases and disorder, as for example pulmonary irritation and toxicity, conjunctivitis and purulent ophthalmitis, mild eye irritation, breast cancer, renal dysfunction, Alzheimer's disease, skin irritation, among others.

Mucins:

Mucins correspond to glycoproteins present in animals and microbes as the main component of mucus. They represent complex substances having heterogeneous characteristics in biological and medical sciences. Their structural properties including the full-length sequence of their main peptide chain and the composition and structure of their branched glycan chains have been clarified only partially. Mucins act as a protecting lining of the mucosa surface, moisturizing material, antimicrobial reagent, lubricant, surfactant, reducer of surface tension, coating material, antifreeze matrix, ion-exchange polymer, amongst other activities (Ushida, K. & Murata, T. “Chapter 4: Materials Science and Engineering of Mucin: A New Aspect of Mucin Chemistry” v. 39, p. 115-159, 2013).

A number of recent research studies based on glycoscience have clearly proved the ability of mucins to perform molecular recognition via their glycan chains, which play the main role in various activities occurring in mucus and around cell surfaces. Typical ligands for glycan chains in mucins are those of the lectin family. This molecular recognition property is the reason for the various functions of mucins mentioned above. Mucins are utilized as a group of efficient materials for controlling the above-mentioned ubiquitous but unique bioactivities of mucins in medical, hygiene, pharmacological, and industrial applications (Ushida & Murata, 2013).

Until 2013, about 20 human mucins have been identified using series names with the header MUC followed by a number. Each MUC is identified in a gene by cDNA cloning with a specific amino acid sequence of the main peptide chain. All of the listed MUC series are roughly separated into two groups: (A) membrane-bound (cell surface) mucins; and (B) secreted (airway) mucins. Examples of membrane-bound (cell surface) mucins are MUC1 and MUC3A. Examples of secreted (airway) mucins are MUC2, MUC5AC, MUC5B, MUC6, MUC8 and MUC19 (gel forming), and MUC7 and MUC9 (nonpolymeric). Each type of MUC is secreted throughout the various mucosal surfaces in the human body. For example, MUC5AC is abundant in both gastric and lachrymal fluids (Ushida & Murata, 2013).

Mucins extracted from other mammals have also been investigated for a long time. They are abundant as commercial materials with relatively reasonable prices. Gastric mucins from pig (porcine gastric mucin, PGM) and rat (RGM), and submaxillary gland mucins from pig (porcine submaxillary gland mucin, PSM), cow (bovine submaxillary gland mucin, BSM), sheep (ovine submaxillary gland mucin, OSM), mouse (MSM), and rat (RSM) are commonly used (Ushida & Murata, 2013).

Glycosylated Polypeptides:

It is known in the art that, depending on the processes and techniques to be applied, the handling and use of mucin can result in the proteic digestion and fragmentation thereof. For instance, during the extraction process of mucine, pepsin can be used. However, said pepsin usually digests and fragmentizes the proteic backbone of full macromolecule of mucin, being obtained as active ingredients glycosylated polypeptides. The state of the art teaches that glycosylated polypeptides from digested and fragmentized mucin are obtained, which show activity for inhibiting microbial biofilm from Pseudomonas aeruginosa (Haley, C. L. et al. “Mucin inhibits Pseudomonas aeruginosa biofilm formation by significantly enhancing twitching motility” Can J Microbiol; 60(3): 155-166; March 2014).

### STATE OF THE ART

The use of mucin in cosmetic topical products for retaining moisture in the skin is described in the state of the art as, for example, in Japanese patent applications JP 62153206 A (published on Jul. 8, 1987, in the name of Kanebo Ltd.), JP 63041412 A (published on Feb. 22, 1988, in the name of Kanebo), JP 03287510 A (published on Dec. 18, 1991, in the name of Pola Chemical Industries and Teikoku Hormone MFG) and JP 10182408 A (published on Jul. 7, 1998, in the name of Kose). However, said prior art references are not focused on deodorant and antiperspirant cosmetics and their effects on the human body and, although aluminum salts are not disclosed therein, said documents do not describe or suggest the benefits and advantages of aluminum-free deodorants and antiperspirants containing mucin.

US patent application US 2015/030661 A1 (published on Jan. 29, 2015, in the name of Massachusetts Institute of Technology) teaches a multilayer film comprising alternating layers of a glycosylated polymer and a lectin, wherein the lectin crosslinks the glycosylated polymers, said glycosylated polymer can be a mucin selected from the group consisting of porcine gastric mucin (purified porcine gastric mucin composed primarily of MUC5AC, MUC2, MUC5B, and MUC6), bovine submaxillary mucin (BSM) or a combination thereof. The multilayer film of US 2015/030661 can be lectin depleted. However, said prior art reference was developed for pharmaceutical purposes, and it is not focused on deodorant and antiperspirant cosmetics and their effects on the human body and, although aluminum salts are not disclosed therein, said documents do not describe or suggest the benefits and advantages of aluminum-free deodorants and antiperspirants containing mucin. US 2015/030661 A1 addresses the ability of the multilayer film to act on the formation of microbial biofilm. In contrast, US 2015/030661 A1 is different from the present invention because it does not use the mucin alone for evaluation of effectiveness to antiperspirant and deodorant activities. Other than that, said prior art document aims at testing glycosylated polymers as mucin in combination with lecithin having main application in the form of a multilayer film acting as a delivery system of active ingredients.

United States patent application US 2004/180027 A1, which was published on Sep. 16, 2004 in the name of Genencor International, Inc., provides a personal care composition comprising an effective amount of a repeat sequence protein polymer and a physiologically acceptable carrier or excipient, wherein said repeat sequence protein polymer comprises a repeating amino acid sequence unit derived from mucin or others. The composition of US 2004/180027 A1 can be used as an antiperspirant. However, said US document describes that the repeat sequence protein polymers used therein are advantageous in providing personal care products when modified with desired chemical agents, as for example aluminum-containing antiperspirant actives. US 2004/180027 A1 differs from the present invention because it does not describe or suggest a mucin polymer in the form of a full glycoprotein or a mixture of glycosylated polypeptides from mucin. Said prior art document uses a polymer obtained in an amount of repetition sequences of amino acids from a protein polymer, as for example mucin, and for this reason it tests a structure formed by the same amino acid sequence and not by all amino acids comprised by the mucin, being therefore chemically and completely different macromolecules.

International publication WO 2014/055127 (published on Apr. 10, 2014, in the name of Katharina Ribbeck) refers to a method of inhibiting virulence of one or more microorganisms, and/or inhibiting one or more microorganisms from attaching to a surface, forming suspended aggregates or a combination thereof, comprising contacting the one or more microorganisms, the surface, or a combination thereof with purified, native, non-human mucin, wherein said non-human mucin can be porcine gastric mucin particularly comprising MUC5AC, MUC2, MUC5B MUC6 or combinations thereof, and wherein said one or more microorganisms can be one or more bacteria, archaea, fungi or a combination thereof. No aluminum salt is used in said WO document. However, although aluminum salts are not disclosed therein, said prior art reference does not describe the benefits and advantages of aluminum-free deodorants and antiperspirants containing mucin nor even the benefit of using mucins in place of aluminum salts in deodorant compositions. Further, there is no description or suggestion in WO 2014/055127 indicating that its object is directed to the axillary microorganisms approached by the present invention. This technical feature is relevant, as it is known in the art that biofilm formation has significant particularities among the different species of microorganisms. Moreover, according to this invention, the matrix tested in the in vivo panel is an organic matrix, unlike inorganic matrices as are the culture plates. The effectiveness of mucin in view of axillary matrix also brings technical features of specificity and differentiation for the present invention, which so far have not been obtained in the state of the art.

Thus, it is desirable to provide deodorant and antiperspirant cosmetic products comprising mucin. Particularly, said deodorant and antiperspirant cosmetic products comprising mucin are free of aluminum-containing substances.

### SUMMARY OF THE INVENTION

The present invention relates to aluminum-containing and aluminum-free mucin topical deodorants and antiperspirants. It is an objective of this invention to provide a topical deodorant and/or antiperspirant formulation for suppressing emergence of body odor formation, comprising a mucin and a physiologically acceptable carrier, which is particularly free of aluminum-containing substances. It is a further objective of this invention to provide the use of a mucin in the preparation of a topical deodorant and/or antiperspirant formulation for suppressing emergence of body odor formation, said formulation being particularly free of aluminum-containing substances.

### BRIEF DESCRIPTION OF THE DRAWINGS

FIG. 1 shows mucins that can prevent bacteria from forming biofilms, which are able to prevent the formation of Pseudomonas aeruginosa biofilms on surfaces and maintain the bacteria in the planktonic state.

### DETAILED DESCRIPTION OF THE INVENTION

A thick, well-hydrated coat of mucus fully covering all moist epithelia in the human body is the key to ensure proper lubrication and protection against pathogens. For example, the inventors verified that healthy mucins, arranged as a 3D gel, can suppress a range of virulence traits across several microbial species, showing mucins have powerful capabilities of regulating microbial behavior. For example, mucins can prevent Pseudomonas aeruginosa from colonizing a surface and from forming potentially deleterious biofilms. Moreover, mucins can prevent the yeast Candida albicans from adhering to an underlying substrate, and moreover from switching from the benign and non-infectious yeast form into the potentially pathogenic filamentous form.

One advantageous feature of mucins is that they suppress microbial virulence without killing the microbes, implying that their presence will not select for the emergence of resistance. Hence, mucins are ideal natural components for using within cleansing, hygiene and cosmetic products that aim at preventing microbial infections or regulating microbial pathogenicity without altering the naturally complex microflora of the human body's surfaces. The inventors of the present invention researched and found that the use of mucins is of high value in products that regulate bacterial populations or prevent the formation of pathogenic biofilms for topical treatments.

The mucus barrier is a vital part of our body, and any disturbance in its function can result in an increased susceptibility to pathogens. Indeed, several important diseases such as cystic fibrosis, inflammatory bowel diseases and dry eye syndrome are correlated with a defective mucus barrier. Decrease in hydration and protective efficiency is also commonly noticed with oral diseases. The underlying reasons for this are mostly unknown, but are very likely to be a result of both insufficient hydration and poor mucus production.

One object of the present invention is to develop novel and nature-inspired strategies for suppressing the emergence of body odor formation. The underarm pit plays an important role in the generation of body odor. Odor is generated from secretions of the apocrine sweat glands, which are primarily contaminated by Coryneform and Staphylococcus bacteria. One potential strategy for suppressing body odor formation is to apply topically to the armpit hydrated mucin polymers that can suppress a range of microbial virulence traits, including the colonization of an underlying epithelium and biofilm formation, as well as favor growth of beneficial microbes that stabilize the microflora in the armpit.

The inventors researched that natively purified mucins can efficiently suppress biofilm formation and other virulence factors in a range of microbes, including Pseudomonas aeruginosa, E. coli, Candida albicans, and Streptococcus mutans. They found that native mucins will also limit the virulence phenotypes in microbes responsible for odor formation by Coryneform and Staphylococcus bacteria. One specific goal of this invention is to demonstrate the behavior of the selected microbes C. jeikeium, C. striatum and S. haemolyticus in a mucin environment. These organisms colonize the underarm epithelium and can cause body odor formation. Specifically, the present invention 1) characterized the influence of native mucins on the behavior of these selected microbes, and 2) studied the influence of mucins on multispecies interactions between bacteria that are responsible for the formation of bad odor and antagonistic bacteria that can suppress their growth.

As described above, depending on the handling and use of mucin, digestion and fragmentation of the proteic backbone of full macromolecule of mucin can occur. Thus, glycosylated polypeptides derived from said digested and fragmentized mucin can be obtained as active ingredients. According to the present invention, said glycosylated polypeptides present cosmetic activities, particularly deodorant and/or antiperspirant activities. For the purposes of this invention, the mucins used herein can be selected from full mucin macromolecules, digested and fragmentized mucins (glycosylated polypeptides therefrom) and combinations thereof.

The mucin used in the present invention prevents or inhibits the adhesion of microorganisms to underlying surfaces, in order to target microorganisms' virulence and to suppress same. In the human body, mucin is employed for suppressing body odor formation that occurs as a result of suppressing a range of microbial virulence traits. Said microorganisms can be bacteria, archaea, fungi or a combination thereof.

For the purposes of the present invention, mucin is selected from the group consisting of porcine gastric mucins (PGM), porcine submaxillary gland mucins (PSM), rat gastric mucins (RGM), bovine submaxillary gland mucins (BSM), ovine submaxillary gland mucins (OSM), mouse submaxillary gland mucins (MSM), rat submaxillary gland mucins (RSM), purified native mucin, hydrated mucin polymer, mucin obtained or derived from fish, and mixtures thereof. Particularly, said mucin is mucin type II (or MUC Type II), mucin type III (or MUC Type III) MUC5AC, purified native mucin, PGM Type II, PGM Type III, Sigma Mucin Type II, Sigma Mucin Type III, hagfish slime mucin, and mixtures thereof.

The present invention is particularly free of aluminum-containing substances. Another object of the present invention is to provide a deodorant and/or antiperspirant that, in addition to comprising mucin, it does not contain any aluminum species. This particular technical feature (absence of aluminum-containing substances) aims at avoiding the damages and disadvantages resulted from the use of aluminum through the body, thus combining the benefits of the mucin active with the prevention of harmful aluminum substances.

It is an embodiment of this invention a topical deodorant and/or antiperspirant formulation for suppressing emergence of body odor formation, comprising a mucin and a physiologically acceptable carrier, which is particularly free of aluminum-containing substances. Particularly, said mucin is present in an amount of 0.5 to 15% by weight of the formulation.

It is a further embodiment of this invention the use of a mucin in the preparation of a topical deodorant and/or antiperspirant formulation for suppressing emergence of body odor formation, said formulation being particularly free of aluminum-containing substances.

The formulations envisaged by the present invention can comprise additional components regularly used in the cosmetic field, being as non-limitative examples: water, perfumes, fragrances, vegetable oils, vegetable essences, sunscreens, emollients, moisturizers, preservatives, surfactants, pH modifiers, vitamins, emulsifiers, lubricants, viscosity modifiers, antioxidants, among others.

Said invention's formulations can be presented as a wash, lotion, cream, emulsion, gel, soap, roll-on, stick, aerosol, and spray to be applied to the body.

### EXAMPLES

### Example 1—Determination of the Effects of Mucins on Selected Individual Odor-Forming Bacterial Species

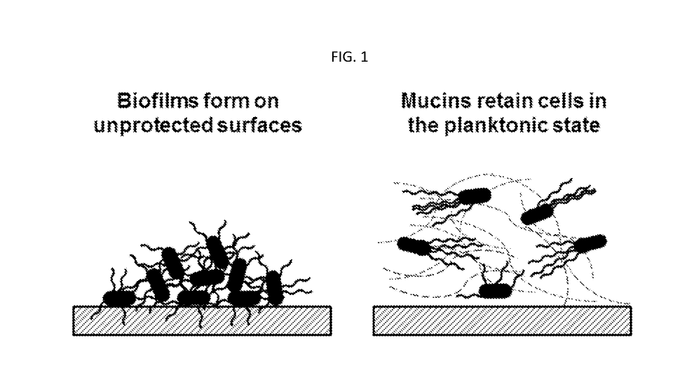

Previous studies that address the effects of mucins on S. mutans behavior used commercially available pig gastric mucins, which differ from native mucins in important ways. Most importantly industrial purification leads to degradation of both the protein backbone and mucin-associated glycans, rendering the molecules non-functional in several ways. The inventors evaluated the effect of mucins on bacterial physiology using purified native mucins from various sources (FIG. 1).

a) Growth Rate in Native and Sigma Aldrich Mucins:

The inventors identified the influence of native mucins on the growth of C. jeikeium, C. striatum and S. haemolyticus in nutrient rich or chemically defined culture media. Growth experiments in nutrient rich media with mucins revealed if native mucins are detrimental to microbial growth. Growth experiments in chemically defined media supplemented with mucins revealed if the microbes can utilize mucins as a nutrient source. A disc diffusion assay was performed to determine the MIC of mucins.

b) Odor Formation and Biofilm Formation in Native Mucins and Sigma Aldrich Mucins Type II:

Biofilm formation contributes to malodor formation and, by analogy, it is likely also involved in odor formation in the armpit. Data obtained by the inventors shown that mucins behave as a defense system to protect the surfaces from colonization by P. aeruginosa, Candida albicans, and S. mutans, and it was found that these biopolymers are also effective against surface colonization of C. jeikeium, C. striatum, C. xerosis and S. haemolyticus. It was investigated surface attachment and biofilm formation in the presence of mucins. Static biofilms and flow-cell biofilms were grown in the presence or absence of mucins. If mucins can inhibit biofilm formation, there is potential to use this biopolymer in formulations that aim to suppress odor formation. According to this invention, mucins are able to adsorb and neutralize secreted small molecules that contribute to odor formation.

The observed timeline was:
Growth rate: 2 months
Surface attachment: 3 months
Biofilm formation: 6 months

The types of mucin that were tested are MUC type II (native) from porcine gastric tissue or from hagfish slime and MUC type III from Sigma Aldrich.

### Example 2—In Vitro Assay

The protocol described in Haley et al. (2014) was also used in this example. The test for biofilm formation with 3 different microorganisms (Staphylococcus haemolyticus, Corynebacterium striatum and Corynebacterium xerosis) was performed as follows: control assay without application of the sample, carrying out readings in times of 8, 24 and 48 hours for each microorganism, in triplicate; assay with two separate samples applied at zero time with microorganisms for verification of biofilm formation with the product applied, carrying out readings in times of 8, 24 and 48 samples, in triplicate. At the same time, it was conducted a test for minimum inhibitory concentration, in duplicate, with 3 microorganisms and 2 products.

### Example 3—In Vivo Assay

It was performed a test for effectiveness regarding odor reduction and modulation of microbiota of the axilla, over 25 volunteers, using a product without antiperspirant and another product having antiperspirant. The metagenomics lasted 4 months, and it was divided into 3 stages: 1) DNA extraction and sample preparation (1 month); 2) sequencing and bioinformatics (2 months); 3) data analysis and preparation of the report (1 month). There were generated results of the qualitative and quantitative identification of axillary microbiota in conditions before and after application of the test products, and analysis' data of the relationship between qualitative and quantitative results of the microbiota with the mal odor phenotype.

### Example 4—Cream Formulation (Consistent Emulsion)

| Phase | Cream (consistent emulsion) | % |
|---|---|---|
| 1 | Aqua or water | 47.6 |
| 1 | Disodium EDTA | 0.1 |
| 2 | Hydroxypropyl startch phosphate | 2 |
| 3 | Ceresin | 3 |
| 4 | Cetearyl alcohol | 7.5 |
| 4 | Ceteareth20 | 1.75 |
| 4 | Dicaprylyl carbonate | 1 |
| 4 | Olus oil/algae oil | 1 |
| 4 | BHT | 0.05 |
| 5 | 2-methyl-5-cyclohexylpentanol | 0.4 |
| 6 | Mucin | 0.5-15 |
| 7 | Talc | 2 |
| 8 | Cyclopentasiloxane and dimethiconol | 1 |
| 8 | Cyclopentasiloxane | 1 |
| 9 | DMDM hydantoin | 0.6 |
| 10 | Fragrancia cotton glaze CL 2 | 1 |

### Example 5—Roll-on Formulation (Fluid Emulsion)

| Phase | Roll-on (fluid emulsion) | % |
|---|---|---|
| 1 | Aqua or water | 53.8 |
| 1 | Disodium EDTA | 0.1 |
| 2 | Hydroxypropyl started phosphate | 2 |
| 3 | PPG15 stearyl ether | 1 |
| 3 | Steareth 2 | 3 |
| 3 | Steareth 21 | 1.1 |
| 3 | BHT | 0.05 |
| 3 | Olus oil/algae oil | 3.8 |
| 4 | Silica dimethyl silylate | 0.15 |
| 4 | 2-methyl-5-cyclohexylpentanol | 0.4 |
| 5 | Mucin | 0.5-15 |
| 6 | DMDM hydantoin | 0.6 |
| 7 | Parfum | 1 |
| 4 | PPG20 methyl glucose ether | 3 |

### Example 6—Aerosol Formulation

| | Aerosol |
|---|---|
| Phase | BIP |
| 1 | PPG14 butyl ether |
| 1 | Disterardimonium hectorite |
| 1 | Propylene carbonate |
| 2 | Cyclopentasiloxane |
| 2 | C12-15 alkyl benzoate |
| 2 | Caprylyl methicone |
| 3 | Mucin |
| 4 | Cyclopentasiloxane (and) cetearyl dimethicone/vinyl |
| | crosspolymer |
| 5 | Olus oil |
| 6 | BHT |
| 7 | 2-methyl-5-cyclohexylpentano |

### Example 7—Spray Formulation

| Phase | Spray | % |
|---|---|---|
| 1 | Alcohol | 70.545 |
| 2 | BHT | 0.05 |
| 2 | Cosmocil | 0.3 |
| 2 | Denatonium benzoate | 0.005 |
| 3 | Zemea | 2 |
| 5 | Lactic acid | 0.1 |
| 5 | Aqua (or) water | 25 |
| 6 | Fragrancia lovely woman body oil | 2 |
| 7 | Mucin | 0.5-15 |

### Example 8—Roll-on Formulation

| | Ingredients | % |
|---|---|---|
| 1 | AQUA | 57.3 |
| 1 | Disodium EDTA | 0.1 |
| 2 | Hydroxypropyl Started Phosphate | 1.5 |
| 3 | STEARETH 21 | 1.1 |
| 3 | STEARETH 2 | 3 |
| 3 | BHT | 0.05 |
| 3 | PPG-15 STEARYL ETHER | 1 |
| 3 | Olus Oil | 3.8 |
| 4 | 2-METHYL 5-CYCLOHEXYLPENTANOL | 0.4 |
| 4 | Silica Dimethyl Silylate | 0.15 |
| 5 | MUCIN | 0.5-15 |
| 6 | DMDM hydantoin | 0.6 |
| 6 | Parfum | 1 |

### Example 9—Deo Cream Formulation

| | Ingredients | % |
|---|---|---|
| 1 | AQUA | 47.6 |
| 1 | Disodium EDTA | 0.1 |
| 2 | Hydroxypropyl Startch Phosphate | 2 |
| 3 | Ceresin | 3 |
| 4 | Cetearyl Alcohol | 7.5 |
| 4 | Ceteareth-20 | 1.75 |
| 4 | Dicaprylyl Carbonate | 1 |
| 4 | Olus Oil | 1 |
| 4 | BHT | 0.05 |
| 5 | 2-METHYL 5-CYCLOHEXYLPENTANOL | 0.4 |
| 6 | MUCIN | 0.5-15 |
| 7 | Talc | 2 |
| 8 | CYCLOPENTASILOXANE AND | 1 |
| | DIMETHICONOL | |
| 8 | Cyclopentasiloxane | 1 |
| 9 | DMDM hydantoin | 0.6 |
| 10 | Parfum | 1 |

### Example 10—Aerosol Formulation

| | Ingredients | % |
|---|---|---|
| | Fase 21639 (30%) | |
| 1 | PPG-14 Butyl ether | 13.33 |
| 1 | BENTONE 38 V CG_DISTEARDIMONIUM | 2.17 |
| | HECTORITE_ELEMENTIS | |
| 1 | Propylene Carbonate | 0.73 |
| 2 | Cyclopentasiloxane | 20.77 |
| 2 | C12-15 Alkyl Benzoate | 14.17 |
| 2 | Caprylyl Methicone | 4.17 |
| 3 | MUCIN | 0.5-15 |
| 4 | Cyclopentasiloxane (and) Cetearyl Dimethicone/Vinyl | 4.17 |
| | Crosspolymer | |
| 5 | Olus Oil | 2.33 |
| 6 | BHT | 0.17 |
| 6 | Parfum | 3.33 |
| 7 | 2-Methyl 5-Cyclohexylpentanol | 1.33 |
| | Fase 21642 (70%) | |
| 1 | BUTANE_ISOBUTANE_PROPANE_70_30 | 100 |

### Example 11—Moisturizing Formulation (for Body)

| | Ingredients | % |
|---|---|---|
| 1 | Butyrospermum Parkii (Shea) Butter | 1 |
| 1 | Caprylic/Capric triglyceride | 2 |
| 1 | Canola Oil | 2 |
| 2 | Dicaprylyl Ether | 2 |
| 2 | ISOHEXADECANO | 2 |
| 2 | Ricinus Communis Seed Oil | 2 |
| 2 | Tocopheryl Acetate | 0.1 |
| 2 | Glycerin | 5 |
| 2 | Olus Oil | 2 |
| 2 | Sphingoceryl WS LS 9859 | 0.2 |
| 3 | Xanthan Gum | 0.2 |
| 3 | Sodium Polyacrylate | 0.9 |
| 4 | Eumulgade CM | 2 |
| 5 | AQUA | 76.1 |
| 5 | Disodium EDTA | 0.1 |
| 6 | Methylisothiazolinone | 0.1 |
| 6 | DMDM hydantoin | 0.5 |
| 6 | LAMESOFT TM BENZ | 0.8 |
| 7 | MUCIN | 0.5-15 |
| 7 | Parfum | 0.5 |
| 8 | ARISTOFLEX AVL | 0.4 |

### Example 12—Moisturizing Formulation (for Feet)

| | Ingredients | % |
|---|---|---|
| 1 | AQUA | 46.2 |
| 2 | Disodium EDTA | 0.1 |
| 3 | Xanthan Gum | 0.1 |
| 3 | Sodium Polyacrylate | 0.5 |
| 4 | Cetyl Lactate and Cetyl Alcohol | 2 |
| 4 | Glyceryl Stearate/Glyceryl Distearate | 2 |
| 4 | Butyrospermum Parkii (Shea) Butter | 10 |
| 4 | Caprylic/Capric triglyceride | 1 |
| 4 | Canola Oil | 2 |
| 4 | Olus Oil | 1 |
| 4 | Manteiga de Cacau Refinada de UIB | 0.3 |
| 5 | AQUA (OR) WATER | 26 |
| 5 | Glycerin | 5 |
| 6 | Eumulgade CM | 1.5 |
| 7 | CYCLOPENTASILOXANE AND DIMETHICONOL | 1 |
| 8 | MUCIN | 0.5-15 |
| 9 | Methylisothiazolinone | 0.1 |
| 9 | DMDM hydantoin | 0.5 |
| 10 | Tocopheryl Acetate | 0.1 |
| 11 | FRAGRÂNCIA LAVMILK BODY MOD13B1C - | 0.5 |
| | 238730 | |

### Example 13—Spray Formulation

| | Ingredients | % |
|---|---|---|
| 1 | Alcohol | 71.5 |
| 1 | MUCIN | 0.5-15 |
| 1 | BHT | 0.05 |
| 1 | BENZOPHENONE-2 | 0.05 |
| 1 | Denatonium Benzoate | 0.005 |
| 1 | CI 42090 | 0.0207 |
| 1 | CI 60730 | 0.135 |
| 2 | PARFUM | 3 |
| 3 | Propanediol | 1 |
| 4 | AQUA | 23.9393 |

### Example 14—Cologne Formulation

| | Ingredients | % |
|---|---|---|
| 1 | Alcohol | 77.329 |
| 1 | MUCIN | 0.5-15 |
| 1 | BHT | 0.05 |
| 1 | BENZOPHENONE-2 | 0.05 |
| 1 | Denatonium Benzoate | 0.005 |
| 2 | Parfum | 10 |
| 3 | AQUA | 12 |
| 4 | SOL ALCOOL 70% CORANTE VERDE (NATURA) | 0.025 |
| | ORGANICO | |
| 4 | SOL. CORANTE 0.1% VIOLETA PURICOLOR 70% | 0.212 |
| | ÁLCOOL | |
| 4 | SOLUÇÃO CORANTE RED 4 0.1% - ÁLCOOL 70° | 0.229 |

1. A topical deodorant and/or antiperspirant formulation for suppressing emergence of body odor formation, comprising a mucin and a physiologically acceptable carrier, which formulation is particularly free of aluminum-containing substances.

2. The formulation according to claim 1, wherein said mucin is present in an amount of 0.5 to 15 wt %.

3. Use of a mucin in the preparation of a topical deodorant and/or antiperspirant formulation for suppressing emergence of body odor formation, said formulation being free of aluminum-containing substances.

4. The formulation according to claim 1, wherein the mucin is selected from full mucin macromolecules, digested and fragmentized mucins (glycosylated polypeptides therefrom) and combinations thereof.

5. The formulation or use according to claim 1, wherein the mucin is selected from the group consisting of porcine gastric mucins (PGM), porcine submaxillary gland mucins (PSM), rat gastric mucins (RGM), bovine submaxillary gland mucins (BSM), ovine submaxillary gland mucins (OSM), mouse submaxillary gland mucins (MSM), rat submaxillary gland mucins (RSM), purified native mucin, hydrated mucin polymer, mucin obtained or derived from fish, and mixtures thereof, preferably mucin type II (or MUC Type II), mucin type III (or MUC Type III) MUC5AC, purified native mucin, PGM Type II, PGM Type III, Sigma Mucin Type II, Sigma Mucin Type III, hagfish slime mucin, and mixtures thereof.

6. The formulation or use according to claim 5, wherein the mucin is MUC Type II, MUC5AC, purified native mucin, and mixtures thereof.

7. The formulation or use according to claim 1, wherein the suppress of body odor formation occurs as a result of suppress a range of microbial virulence traits.

8. The formulation or use according to claim 1, wherein it further comprises one or more cosmetically acceptable components selected from the group consisting of water, perfumes, fragrances, vegetable oils, vegetable essences, sunscreens, emollients, moisturizers, preservatives, surfactants, pH modifiers, vitamins, emulsifiers, and lubricants.

9. The formulation or use according to claim 1, wherein it is in the form of a body wash, lotion, cream, emulsion, gel, soap, roll-on, stick, aerosol, or spray.
